(19) United States
(12) Patent Application Publication
(10) Pub. No.: US 2023/0172829 A1
(43) Pub. Date: Jun. 8, 2023

(54) TREATMENT REGIMEN WITH MINOXIDIL AND CAFFEINE

(71) Applicant: Dr. Kurt Wolff GmbH & Co. KG, Bielefeld (DE)

(72) Inventors: Erik SCHULZE ZUR WIESCHE, Bielefeld (DE); Maike BECKER, Bielefeld (DE); Joern Michael VOELKER, Bielefeld (DE)

(73) Assignee: Dr. Kurt Wolff GmbH & Co. KG, Bielefeld (DE)

(21) Appl. No.: 17/919,955

(22) PCT Filed: Apr. 20, 2021
(86) PCT No.: PCT/EP2021/060166

§ 371 Date: Oct. 19, 2022

(30) Foreign Application Priority Data
Apr. 20, 2020 (EP) 20170357.6

(51) Int. Cl. A61K 8/49; A61K 8/34; A61K 8/67; A61Q 7/00

(52) U.S. Cl. CPC A61K 8/4953; A61K 8/34; A61K 8/675; A61Q 7/00

(57) ABSTRACT

The invention relates to the treatment of hair loss with minoxidil and caffeine.

### FIELD OF THE INVENTION

The present invention is directed to a method for the treatment and/or prevention of hair loss comprising the application of minoxidil and caffeine.

### BACKGROUND OF THE INVENTION

Hair loss, or alopecia, constitutes a concern affecting all humans. Alopecia can be caused by hereditary factors, hormonal factors, surgery, trauma, medical treatments like chemotherapy, or stress.

Due to modern beauty ideals, the search for effective ways for treating or preventing hair loss is an ongoing endeavor.

Only few drugs have been clinically proven to be effective for the prevention of hair loss, such as minoxidil, finasteride, and dutasteride.

The exact mechanism of action of minoxidil is still not fully elucidated but it is believed that the metabolite minoxidil sulfate plays an important role in the proliferation of hair keratinocytes.

Minoxidil is commonly applied topically as an ointment to the scalp, i.e. in the morning and in the evening. Subsequently, the ointment is left in contact with the scalp so that the minoxidil can be absorbed through the skin (leave-on product).

However, commercially available minoxidil solutions commonly contain propylene glycol, which usually results in oily hair that has a fatty and glossy appearance. In addition, dandruff and skin rashes are frequently observed side effects that are experienced during the treatment of hair loss.

Therefore, it was an object of the present invention to provide an administration regimen for the treatment of hair loss that overcomes the above-mentioned drawback while maintaining effectiveness of the treatment. In particular, it was an object of the present invention to provide an administration regimen that prevents fatty appearance of the hair while maintaining effectiveness. It was another object of the invention to provide an administration regimen for the treatment of hair loss that has less side effects while maintaining effectiveness.

In addition, it was a further object of the present invention to provide an administration regimen that results in a synergistic effect regarding the treatment and/or prevention of hair loss.

### SUMMARY OF THE INVENTION

These objects have surprisingly been solved by a non-therapeutic method for the treatment and/or prevention of hair loss comprising the following steps in any order:
(a) topically applying a composition (1) comprising minoxidil to the scalp and leaving the composition (1) in contact with the scalp for 5 to 15 h;
(b) topically applying a composition (2) comprising a surfactant and optionally caffeine to the scalp and leaving the composition (2) in contact with the scalp for 1 to 10 minutes; and at least in case when composition (2) does not comprise caffeine
(c) topically applying a composition (3) comprising caffeine to the scalp and leaving the composition (3) in contact with the scalp for at least 6 h.

It has surprisingly been found that according to the above aspect, minoxidil can be applied only once daily without sacrificing any of its effectiveness and at the same time fatty or glossy appearances of the hair is reduced due to the application of composition (2).

In another aspect, the present invention relates to a combination of a composition (1) comprising minoxidil and a composition (2) comprising caffeine for use in the treatment and/or prevention of hair loss,

wherein administration comprises the following steps in any order:
(a) topically applying a composition (1) comprising minoxidil to the scalp and leaving the composition (1) in contact with the scalp for 5 to 15 h; and
(b) topically applying a composition (2) comprising a surfactant and optionally caffeine to the scalp and leaving the composition (2) in contact with the scalp for 1 to 10 minutes; and at least in case when composition (2) does not comprise caffeine
(c) topically applying a composition (3) comprising caffeine to the scalp and leaving the composition (3) in contact with the scalp for at least 6 h.

The present invention also relates to a kit of parts comprising a composition (1) comprising minoxidil, a composition (2) comprising a surfactant and/or caffeine and at least in case when composition (2) does not comprise caffeine, a composition (3) comprising caffeine, and optionally a leaflet.

In yet another aspect, the invention relates to the use of the kit of parts for the cosmetic treatment and/or prevention of hair loss.

In another aspect, the intention relates to the kit of parts for use in the treatment and/or prevention of hair loss.

### DETAILED DESCRIPTION

The following definitions are relevant in connection with the embodiments of the present invention.

The meaning of the term “comprising” is to be interpreted as encompassing all the specifically mentioned features as well optional, additional, unspecified ones, whereas the term “consisting of” only includes those features as specified. Therefore, “comprising” includes as a limiting case the meaning of “consisting of”.

The term “weight-%” or “wt.-%” is to be understood to refer to the weight amount of the component relative to the total weight amount of the respective composition, if not specified otherwise. The term “volume-%” or “vol.-%” is to be understood to refer to the volume amount of the component relative to the total volume amount of the respective composition, if not specified otherwise.

Preferred embodiments according to the invention are defined hereinafter. The preferred embodiments are preferred alone or in combination. Further, it is to be understood that the following preferred embodiments refer to all aspects of the present invention, i.e. the non-therapeutic method, the combination for use, the kit of parts, the use of the kit of parts and the kit of parts for use.

In an embodiment, the composition (1) comprises 0.1 to 5 wt.-% minoxidil. It is preferred that composition (1) comprises 1 to 5 wt.-% minoxidil. In a particularly preferred embodiment, the composition (1) comprises 2, 2.5, 3, 3.5, 4, 4.5 or 5 wt.-% minoxidil.

In an embodiment, the composition (1) is a gel, a foam or a solution, preferably a solution.

In case the composition (1) is a foam, composition (1) may comprise cetyl alcohol, stearyl alcohol, and butylated hydroxytoluene. In case composition (1) is a foam, it does typically not contain a glycol compound, such as propylene glycol.

In case the composition (1) is a solution, composition (1) may comprise an alcohol selected from the group consisting of ethanol, isopropanol, n-propanol, and mixtures thereof, and/or a glycol selected from the group consisting of propylene glycol, dipropylene glycol, 1,3-butylene glycol, pentylene glycol, dipentylene glycol, and mixtures thereof and/or water. It is preferred that the alcohol is ethanol and/or isopropanol. It is preferred that the glycol is propylene glycol. It is even more preferred that the composition (1) comprises 1 to 5 wt.-% minoxidil, ethanol, propylene glycol, and water.

In an embodiment, the composition (1) comprises 10 to 80 vol.-% ethanol, 20 to 50 vol.-% ethanol, or 25 to 35 vol.-% ethanol. It is preferred that the composition (1) comprises 25 to 35 vol.-% ethanol. In an embodiment, the composition (1) comprises 10 to 80 vol.-%, 20 to 70 vol.-% or 40 to 60 vol.-% propylene glycol. In a preferred embodiment, the composition (1) comprises 1 to 5 wt.-% minoxidil, 10 to 80 vol.-% propylene glycol and 10 to 80 vol.-% ethanol, and water.

In an embodiment, the composition (1), the composition (2), and/or the composition (3) comprises a preservative independently selected from the group consisting of benzoic acid, benzyl alcohol, chlorocresol, paraben esters, sorbic acid, ascorbic acid and salts thereof, sodium metabisulfite, chelating agents, citric acid, salicylic acid, and mixtures thereof.

In an embodiment, the composition (1), the composition (2), and/or the composition (3) comprises a pH modifier independently selected from the group consisting of diethanolamine, lactic acid, monoethanolamine, sodium hydroxide, sodium phosphate, triethanolamine, and mixtures thereof.

In an embodiment, the hair loss is hereditary hair loss. Hereditary hair loss commences after a certain age and may therefore also be called hair loss due to ageing. Androgenic alopecia, often also called androgenetic alopecia, is the most common hereditary hair loss and it was believed that it is caused by a high free concentration of androgens, such as DHT (dihydrotestosterone). Androgenic alopecia is gender-specific, resulting in male or female pattern hair loss. In another embodiment, the hair loss is alopecia areata, scarring alopecia, chemotherapyinduced alopecia, chronic telogen effluvium, and/or hair shaft disorder.

As the compositions (1) and (3) are left in contact with the scalp for an extended time period, they are also referred to as leave-on compositions in contrast to composition (2) which is a rinse-off composition, as it is typically rinsed off after 1 to 10 minutes. Composition (2) is commonly applied after the composition (1). The purpose of applying the composition (2) in step (b) is to remove or wash out the composition (1) from the hair and from the scalp.

In an embodiment, the composition (2) comprises a surfactant and 0.01 to 10 wt.-% of caffeine. It is preferred that the composition (2) is a shampoo. It is more preferred, that the composition (2) is a caffeine shampoo comprising 0.05 to 10, 0.1 to 5, or 0.2 to 1 wt.-% caffeine. In an embodiment, composition (3) comprises 0.05 to 10, 0.1 to 5, or 0.2 to 1 wt.-% caffeine.

The sequential treatment with minoxidil and caffeine is an essential part of the present invention and caffeine should be applied to the scalp either as part of applying composition (2) as rinse-off caffeine shampoo and/or as part of applying composition (3) as a leave-on caffeine lotion. In case the composition (2) does not contain caffeine, the step (c) is a mandatory, i.e. not an optional step. In contrast, step (c) may also be performed as an additional, optional step in case composition (2) contains caffeine.

In an embodiment, the composition (3) does not contain a surfactant. In another embodiment, the composition (2) has a viscosity of 2000 to 6000 mPa·s, whereas the composition (3) has a viscosity of 1 to 100 mPa·s.

The viscosity is measured at 20° C. and a shear rate of 10/s with a rheometer using DIN 53019-1:2008-09.

Thus composition (2) and composition (3) differ from each other in that composition (2) is a rinse-off product and composition (3) is a leave-on product, and in that composition (2) has a higher viscosity than composition (3).

In an embodiment, the composition (2) and/or the composition (3) comprise a further additional active ingredient independently selected from the group consisting of vitamins, flavonoids, terpenes, saccharides, plant extracts, and mixtures thereof.

Non-limiting examples of vitamins are niacin, biotin, vitamin E (also referred to as tocopherol), vitamin A, and vitamin C. Vitamins are antioxidants and can have various additional useful properties.

Non-limiting examples of flavonoids are apigenin, luteolin, tangeritin, quercetin, kaempferol, myricetin, fisetin, galangin, isorhamnetin, pachypodol, rhamnazin, pyranoflavonols, and furanoflavonols.

Non-limiting examples of terpenes are farnesol, menthol, limonen, squalene, camphor, pinene, eucalyptol, retinol, geraniol, linalool, carvone and thymol.

Non-limiting examples of saccharides are saccharides derived from starch, sorbitol and maltitol.

Plant-extracts can have multiple purposes and are i.a. useful as natural antioxidant, antiaging, emollient, anti-inflammatory, anti-irritant, and/or antimicrobial component. Non-limiting examples of plant extracts are plant oils, such as soy oil, sunflower oil, almond oil, grape oil, avocado oil, castor oil, glycine soja oil, jojoba oil and oils from Areca catechu, Crocus sativus, Curcuma longa, green tea, Crataevea murula, Rosmarinus Officinalis, buckwheat seeds, Emblica officinale, Ginkgo biloba, Centella asiatica, Psoraliea corylifolia, Citrus limonum, Aloe Vera, Matricaria recutita, Thea viridis, Vitis vinifera, Daucus carota, Lycopersicon esculentum, Allium sativum and Hamamelis virginiana.

In a preferred embodiment, the composition (2) comprises 0.01 to 2 wt.-% niacinamide. In another preferred embodiment, the composition (2) comprises 0.01 to 2 wt.-% biotin.

In another embodiment, the composition (2) comprises hydrolyzed wheat protein. In yet another embodiment, the composition (2) comprises an amino acid, preferably arginine or leucine.

In an embodiment, the surfactant in composition (2) is selected from the group consisting of non-ionic surfactants, anionic surfactants, cationic surfactants, amphoteric surfactants and mixtures thereof. In another embodiment, the composition (2) comprises 1 to 4 vol.-% of a non-ionic surfactant.

Surfactants are molecules that comprise a hydrophilic and a hydrophobic moiety at opposite ends. Suitable surfactants are described in “Cosmetic Formulation of Skin Care Products”, Draelos et al., Taylor & Francis Group, 2006.

In an embodiment, the non-ionic surfactant is selected from the group consisting of C₄-C₂₆ fatty alcohol ethoxylates, alkylphenol ethoxylates, ethoxylated amines/amides, glycerol C₄-C₂₆ fatty acid amides, glycerol C₄-C₂₆ fatty acid esters, sorbitol fatty acid esters, and mixtures thereof. The prefix “Cₓ-C_{y}” denotes the number of carbon atoms of the respective functional group. Non-limiting examples of a C₄-C₂₆ fatty alcohol ethoxylates are pentaethylene glycol monododecyl ether (also referred to as C12E5), hexaethylene glycol monododecyl ether (C12E6), heptaethylene glycol monododecyl ether (C12E7), and octaethylene glycol monodecyl ether (C12E8). Non-limiting examples of alkylphenol ethoxylates are Triton X-100® (octyl phenol ethoxylate) and Nonidet P-40® (4-nonylphenol ethoxylate). Non-limiting examples of ethoxylated amines/amides are polyethoxylated tallow amine, cocamide monoethanolamine, and cocamide diethanolamine. Non-limiting examples of glycerol C₄-C₂₆ fatty acid esters are glycerol monostearate and glycerol monolaurate. Non-limiting examples of sorbitol fatty acid esters are sorbitan monolaurate, sorbitan monostearate and sorbitan tristearate.

Anionic surfactants dissociate in water to yield an anionic surfactant in water. Anionic surfactants useful herein include alkyl sulfates and alkyl ether sulfates. These materials have the respective formulae ROSO₃M and RO(C₂H₄O)ₓSO₃M wherein R is a C₄-C₂₆ alkyl or C₄-C₂₆ alkenyl, x is 1 to 10, and M is a water-soluble cation such as ammonium, sodium, potassium and triethanolamine. The alkyl ether sulfates useful in the present invention are condensation products of ethylene oxide and monohydric alcohols having about 4 to about 26 carbon atoms. Preferably, R is C₁₄-C₁₆ alkyl or C₁₄-C₁₈ alkenyl in both the alkyl and alkyl ether sulfates. The alcohols can be derived from fats, e.g., coconut oil or tallow, or can be synthetic. Lauryl alcohol and straight chain alcohols derived from coconut oil are preferred herein. Such alcohols are reacted with 1 to 10, and especially 3, molar proportions of ethylene oxide and the resulting mixture of molecular species, having, for example, an average of 3 moles of ethylene oxide per mole of alcohol, is sulfated and neutralized. Specific examples of alkyl ether sulfates of the present invention are sodium coconut alkyl trioxyethylene sulfate; lithium tallow alkyl trioxyethylene sulfate; sodium tallow alkyl hexaoxyethylene sulfate; and sodium lauryl ether sulfate. Additional examples of anionic surfactants which come within the terms of the present invention are the reaction product of fatty acids esterified with isethionic acid and neutralized with sodium hydroxide where, for example, the fatty acids are derived from coconut oil; sodium or potassium salts of fatty acid amides of methyl tauride in which the fatty acids, for example, are derived from coconut oil.

Cationic surfactants comprise primary, secondary tertiary amines, such as octenidine dihydrochloride, and quaternary ammonium salts, such as cetrimonium bromide, cetlypyridinium chloride, benzalkonium chloride, benzethonium chloride, dimethyldioctadecylammonium chloride and dioctadecyldimethylammonium bromide.

Amphoteric surfactants have an anionic and a cationic functional group. Examples of amphoteric surfactants are derivatives of aliphatic secondary and tertiary amines in which the aliphatic radical can be straight chain or branched and wherein one of the aliphatic substituents contains from about 8 to about 18 carbon atoms and one contains an anionic water solubilizing group, e.g., carboxy, sulfonate, sulfate, phosphate, or phosphonate. Specific examples of amphoteric surfactants are sodium 3-dodecylaminopropionate, sodium 3-dodecylaminopropane sulfonate, and N-alkyltaurines such as the one prepared by reacting dodecylamine with sodium isethionate. Particularly preferred amphoteric surfactants are betaines such as, for example, coco-amidopropyl betaines. The amphoteric surfactant component may be present in the composition in an amount ranging from about 5 to about 15% by weight, and preferably from about 9 to about 12% by weight, based on the weight of the composition.

In an embodiment, the composition (2) and/or the composition (3) comprise a diluent independently selected from the group consisting of water, glycerin, a glycol, sorbitol, ethanol, triacetin and mixtures thereof. In an embodiment, the composition (2) and the composition (3) do not contain a glycol.

In an embodiment, the kit of parts further comprises an application device for applying the composition (1), the composition (2) and/or the composition (3) to the scalp. The application device is suitable as a means for applying any one of the compositions. The application device may be an integral part of the container in which the composition is provided in or can be an external device, such as a suitable pipette or a contact applicator that is adapted to be attached to an opening of the container in which any one of the compositions is provided in. The application device can also be a pumpspray applicator. It is particularly preferred that the application device contains a means for determining the amount of the composition that is applied to the scalp.

The following formulation of a composition (2) and composition (3) are examples of a typical formulation:

In a typical example, the composition (2) comprising caffeine contains 4.0 wt.-% sodium lauryl sulfate, 5.0 wt.-% sodium laureth sulfate, 1.2 wt.-% sodium lauroyl glutamate, 1.0 wt.-% hydroxypropyltrimonium hydrolyzed wheat, 3.0 wt.-% disodium laureth sulfosuccinate, 0.3 wt.-% biotin, 0.2 wt.-% tocopherylacetate, 0.2 wt.-% citric acid, 1 wt.-% caffeine, 0.3 wt.-% fragrance, 0.2 wt.-% potassium sorbate, 0.5 % salicylic acid, 0.05 wt.-% niacinamide, and purified water.

In a typical example, the composition (3) contains 40.0 wt.-% ethanol, 0.05 wt.-% tocopheryl acetate, 0.3 wt.-% PEG-40 hydrogenated castor oil, 0.3 wt.-% caffeine, 0.2 wt.-% glycine soya oil, 0.3 wt.-% panthenol, 0.2 wt.-% fragrance, 0.1 wt.-% menthol, 0.05 wt.-% niacinamide, and purified water.

In an embodiment, the administration consists of the following steps in any order:
(a) topically applying a composition (1) comprising minoxidil to the scalp and leaving the composition (1) in contact with the scalp for 5 to 15 h;
(b) topically applying a composition (2) comprising a surfactant and optionally caffeine to the scalp and leaving the composition (2) in contact with the scalp for 1 to 10 minutes; and at least in case when composition (2) does not comprise caffeine
(c) topically applying a composition (3) comprising caffeine to the scalp and leaving the composition (3) in contact with the scalp for at least 6 h.

In an embodiment, the steps are performed in sequential, alphabetical order. It is preferred that step (a) is performed in the evening and step (b) and step (c) are performed the next morning. Thus, the steps (a) to (c) are performed once within 24 hours. It is preferred that the composition (1) comprising minoxidil is applied only once daily to the scalp (and not twice daily).

Thus, in a particularly preferred embodiment, the administration comprises
(a) topically applying a composition (1) comprising minoxidil to the scalp in the evening and leaving the composition (1) in contact with the scalp overnight; and
(b) topically applying a composition (2) comprising a surfactant and/or caffeine to the scalp in the morning and leaving the composition (2) in contact with the scalp for 1 to 10 minutes; and at least in case when composition (2) does not comprise caffeine
(c) topically applying a composition (3) comprising caffeine to the scalp and leaving the composition (3) in contact with the scalp for at least 6 h.

In an even more preferred embodiment, the administration comprises
(a) topically applying a composition (1) comprising minoxidil to the scalp in the evening and leaving the composition (1) in contact with the scalp overnight; and
(b) topically applying a composition (2) comprising 1 to 4 vol.-% of a non-ionic surfactant and 0.01 to 10 wt.-% of caffeine to the scalp in the morning and leaving the composition (2) in contact with the scalp for 1 to 10 minutes.

In an even more preferred embodiment, the administration comprises
(a) topically applying a composition (1) comprising minoxidil to the scalp in the evening and leaving the composition (1) in contact with the scalp overnight; and
(b) topically applying a composition (2) comprising 1 to 4 vol.-% of a non-ionic surfactant and not comprising any caffeine to the scalp in the morning and leaving the composition (2) in contact with the scalp for 1 to 10 minutes; and
(c) topically applying a composition (3) comprising 0.01 to 5 wt.-% caffeine to the scalp and leaving the composition (3) in contact with the scalp for at least 6 h.

Most preferably, the administration regimen of steps (a) to (c) in accordance with the present invention is conducted once daily (within 24 hours) over a non-limited period of time, preferably for at least several months. In other words, the administration regimen of steps (a) to (c) in accordance with the present invention is repeated every day for the total course of the intended and/or prescribed treatment of the hair and scalp with the ingredients minoxidil and caffeine. Using this administration regimen, minoxidil is applied only once daily which overcomes the disadvantages known from the prior art while the overall effect on hair growth is not reduced.

### EXAMPLES

### Example 1: Treatment With Minoxidil Solution and Caffeine Shampoo (Rinse-Off)

A minoxidil solution containing 5 wt.-% minoxidil, 30 vol.-% ethanol, 50 vol.-% propylene glycol and water was provided.

A composition (2) being a caffeine shampoo containing 4.0 wt.-% sodium lauryl sulfate, 5.0 wt.-% sodium laureth sulfate, 1.2 wt.-% sodium lauroyl glutamate, 1.0 wt.-% hydroxypropyltrimonium hydrolyzed wheat, 3.0 wt.-% disodium laureth sulfosuccinate, 0.3 wt.-% biotin, 0.2 wt.-% tocopherylacetate, 0.2 wt.-% citric acid, 1 wt.-% caffeine, 0.3 wt.-% fragrance, 0.2 wt.-% potassium sorbate, 0.5 % salicylic acid, 0.05 wt.-% niacinamide, and purified water was provided.

Subjects applied 1 mL of minoxidil solution in the evening and applied the caffeine shampoo of composition (2) to scalp on the next morning. The composition (2) was left in contact with the scalp for 1 to 10 minutes and subsequently rinsed off.

### Example 2: Treatment With Minoxidil Solution, Surfactant Shampoo (Rinse Off) And Caffeine Solution (Leave-On)

The treatment in Example 2 was identical to that in Example 1, except that the composition (2) of Example 1 did not contain caffeine and after rinsing off the composition (2), a caffeine solution as composition (3) containing 40.0 wt.-% ethanol, 0.05 wt.-% tocopheryl acetate, 0.3 wt.-% PEG-40 hydrogenated castor oil, 0.3 wt.-% caffeine, 0.2 wt.-% glycine soya oil, 0.3 wt.-% panthenol, 0.2 wt.-% fragrance, 0.1 wt.-% menthol, 0.05 wt.-% niacinamide, and purified water was applied to the scalp and left in contact with the scalp until the evening.

### Comparative Example 1: Treatment With Minoxidil Solution Twice Daily Without Caffeine

In Comparative Example 1, subjects applied 1 mL of the minoxidil solution of Example 1 in the evening and applied a shampoo of composition (2), being identical to that used in Example 1 but not containing caffeine, to scalp on the next morning. The composition (2) was left in contact with the scalp for 1 to 10 minutes and subsequently rinsed off. Subsequently 1 mL of the minoxidil solution was applied to the scalp and left in contact with the scalp until the evening.

Thus, the minoxidil solution was applied twice daily without applying a caffeine containing shampoo and/or caffeine solution.

### Comparative Example 2: Treatment With Minoxidil Solution Further Containing Caffeine Twice Daily

In Comparative Example 1, subjects applied 1 mL of the minoxidil solution of Example 1 additionally containing 1 wt.-% caffeine in the evening and applied a shampoo of composition (2), being identical to that used in Example 1 but not containing caffeine, to scalp on the next morning. The composition (2) was left in contact with the scalp for 1 to 10 minutes and subsequently rinsed off. Subsequently, 1 mL of the minoxidil solution additionally containing 1 wt.-% caffeine was applied to the scalp and left in contact with the scalp until the evening.

Thus, a minoxidil solution further comprising caffeine was applied twice daily without applying a caffeine containing shampoo and/or caffeine solution.

The subjects treated according to Example 1 showed less fatty hair compared to subjects treated according to Comparative Example 1 or Comparative Example 2. After 3 months, no reduction in the prevention of hair loss could be observed between subjects treated according to Example 1 and subjects treated according to Comparative Example 1 or Comparative Example 2.

The subjects treated according to Example 2 showed less fatty hair compared to subject treated according to Comparative Example 1 or Comparative Example 2. After 3 months, no reduction in the prevention of hair loss could be observed between subjects treated according to Example 2 and subjects treated according to Comparative Example 1 or Comparative Example 3.

1-15. (canceled)

16. A method for the treatment and/or prevention of hair loss comprising the following steps in any order:
(a) topically applying a first composition comprising minoxidil to a scalp of a subject and
leaving the first composition in contact with the scalp for 5 to 15 h;
(b) topically applying a second composition comprising a surfactant and optionally caffeine to the scalp and leaving the second composition in contact with the scalp for 1 to 10 minutes; and at least in case when the second composition does not comprise caffeine
(c) topically applying a third composition comprising caffeine to the scalp and leaving the third composition in contact with the scalp for at least 6 h.

17. The method of claim 16, wherein the first composition comprises 0.1 to 5 wt.-% minoxidil.

18. The method of claim 16, wherein the first composition comprises an alcohol selected from the group consisting of ethanol, isopropanol, n-propanol, and mixtures thereof, and/or wherein the first composition comprises a glycol selected from the group consisting of propylene glycol, dipropylene glycol, 1,3-butylene glycol, pentylene glycol, dipentylene glycol, and mixtures thereof.

19. The method of claim 16, wherein the first composition comprises 1 to 20 vol.-% propylene glycol and 20 to 80 vol.-% ethanol.

20. The method of claim 16, wherein the second composition comprises 0.01 to 10 wt.-% of caffeine.

21. The method of claim 16, wherein the second composition comprises a further additional active ingredient selected from the group consisting of vitamins, flavonoids, terpenes, saccharides, plant extracts, and mixtures thereof.

22. The method of claim 16, wherein the second composition comprises 0.01 to 2 wt.-% niacinamide and/or 0.01 to 2 wt.-% biotin.

23. The method of claim 16, wherein the second composition comprises 1 to 4 vol.-% of a non-ionic surfactant.

24. The method of claim 16, wherein the second composition comprises a diluent selected from the group consisting of water, glycerin, a glycol, sorbitol, ethanol, triacetin and mixtures thereof.

25. A combination of a first composition comprising minoxidil and a second composition comprising caffeine for use in the treatment and/or prevention of hair loss,
wherein the combination is able to be administered in the following steps in any order:
(a) topically applying the first composition comprising minoxidil to the scalp and leaving the first composition in contact with the scalp for 5 to 15 h; and
(b) topically applying the second composition comprising a surfactant and optionally caffeine to the scalp and leaving the second composition in contact with the scalp for 1 to 10 minutes; and at least in case when the second composition does not comprise caffeine
(c) topically applying a third composition comprising caffeine to the scalp and leaving the third composition in contact with the scalp for at least 6 h.

26. The combination of claim 25, wherein the first composition comprises 0.1 to 5 wt.-% minoxidil.

27. The combination of claim 25, wherein the first composition comprises an alcohol selected from the group consisting of ethanol, isopropanol, n-propanol, and mixtures thereof, and/or wherein the first composition comprises a glycol selected from the group consisting of propylene glycol, dipropylene glycol, 1,3-butylene glycol, pentylene glycol, dipentylene glycol, and mixtures thereof.

28. The combination of claim 25, wherein the first composition comprises 1 to 20 vol.-% propylene glycol and 20 to 80 vol.-% ethanol.

29. The combination of claim 25, wherein the second composition comprises 0.01 to 10 wt.-% of caffeine.

30. The combination of claim 25, wherein the second composition comprises a further additional active ingredient selected from the group consisting of vitamins, flavonoids, terpenes, saccharides, plant extracts, and mixtures thereof.

31. The combination of claim 25, wherein the second composition comprises 0.01 to 2 wt.-% niacinamide and/or 0.01 to 2 wt.-% biotin.

32. The combination of claim 25, wherein the second composition comprises 1 to 4 vol.-% of a non-ionic surfactant.

33. The combination of claim 25, wherein the second composition comprises a diluent selected from the group consisting of water, glycerin, a glycol, sorbitol, ethanol, triacetin and mixtures thereof.

34. A kit comprising a first composition comprising minoxidil, a second composition comprising a surfactant and/or caffeine, and at least in case when second composition does not comprise caffeine, a third composition comprising caffeine, and optionally a leaflet.

35. The kit of claim 34, further comprising an application device for applying the first composition, the second composition, and/or the third composition to a scalp of a subject.

36. A method for treating and/or preventing hair loss comprising applying the first composition, the second composition, and/or the third composition of the kit of claim 34 to a scalp of a subject.

37. The kit of claim 34, wherein the first composition comprises 0.1 to 5 wt.-% minoxidil.

38. The kit of claim 34, wherein the first composition comprises an alcohol selected from the group consisting of ethanol, isopropanol, n-propanol, and mixtures thereof, and/or wherein the first composition comprises a glycol selected from the group consisting of propylene glycol, dipropylene glycol, 1,3-butylene glycol, pentylene glycol, dipentylene glycol, and mixtures thereof.

39. The kit of claim 34, wherein the first composition comprises 1 to 20 vol.-% propylene glycol and 20 to 80 vol.-% ethanol.

40. The kit of claim 34, wherein the second composition comprises 0.01 to 10 wt.-% of caffeine.

41. The kit of claim 34, wherein the second composition comprises a further additional active ingredient selected from the group consisting of vitamins, flavonoids, terpenes, saccharides, plant extracts, and mixtures thereof.

42. The kit of claim 34, wherein the second composition comprises 0.01 to 2 wt.-% niacinamide and/or 0.01 to 2 wt.-% biotin.

43. The kit of claim 34, wherein the second composition comprises 1 to 4 vol.-% of a non-ionic surfactant.

44. The kit of claim 34, wherein the second composition comprises a diluent selected from the group consisting of water, glycerin, a glycol, sorbitol, ethanol, triacetin and mixtures thereof.
